SEQUENCE LISTING (3 sequences)

SEQ ID NO: 1 - DNA, 3973 bp, Homo sapien
gtctatcacgaacaccatatgccaatacatttaacaatttagaggaaatgatcaaattccttgtgttaaaatataaacttttccacattaaaatttcaagctggccaggcgtggtggctcacacctgtaatcccatcactttgggaggccaaggtgggtggattgcctgagctcaggagttcgagaccacccaggcaacatggtgaaacccagtctctactaaaaaaatacaaaaaaattaactgggtgtgatggcacatgcctctagtcccagctacttgggggccgaggcaggagaatctcttgagccctagaggtgaaggctgcagtgagccaagattgtgccactgcactccagcttgggctacagagtgagactctgtctcaaaaaaaaaaaaaaaaaaaaaaaaaaaaaaaaaaaaaattaaggtgttccagcagtgattcataaattaagcagctacaaaccagaaatggtctggggctctgttagagggggttgaagggacacagagcaaagaaattgtttcattggttaaagtttgagtgtttgccttatttggactatccaagtgagaaatttatgacaatataaccaatgtcccagttagccaactgtgattggctaaactaagtttcattttcctcatgagggtgttacagctacatcagtctaatggcctaccaattattttaacacttgagaagcacaatttatcaaaactgattaaagattaaaagtcagaatagctttatatctaattgagaaattgaattcattataaagaatcttcaaacaaggaaaatgtcaagcccaaatggttttattagtgaattctgttaaacatttaaggggaaaatgataccaatctgaaacaaactcttgctttatgagactagcataactctgatatcaaaacctgataaacacattacaagaaaataaaattattgactaatattcctcatgaacacagacacaaaagtatttatgaaaatactcatttggtcatgatatagcatcctttgtatctaaaaccaaatgagatttatcccaggaatggtaggttgggtgaacgttcaaaaattgatcagtgaaatttggtatgttaacagaatatatgatcacctcaataaatgcaggaaaaaacatttgaaaaattcaacacccatgcattataaaaactcagcaaactaggaataaaagagaacttcatcaatctgataaaaggtatctaagaaaaatttacagctaccatattatttaaagtgattattattacattgatgatttccccactgaaattagcaataaagtaatggtgtcaactgtcaccacttttattctattttctataggaggctctaggaggtaccataataaagaaaggaggcaaaaagcatgaagaccggaaagaaagaagtaaaactggctgggcatggtggctcatgtctgtaatcccagcactttgagaggccgagatgggcgatcgcttgagttcaggagtttgagaccagcctgggcatcagggcgaaaccccatctctacaaaaaaatacaaaaattagctgggtgtagtggtgtgcacctgtggtcctagctactcaggaggctgcagtggaaggatccttgagcctgggaggtggaggctgcagcagcctggttgacaaaagtgagaccctgtctcaaaaagaaaaactataatgaaaggaaataaaaggcataaagattggaaaggaagaagttaaagtatgtattcacaaatgacatcattatttatctagaaaacttagaggaatttacagaacaattactagaacaaataagtgaagttagcaatgttgcatgttttaaggtcaaaaaacaaaaatcacttgtatttctttttttgtgtgtgtgtgtgagatggagtctcactctgtcgcccaggctggagtgcagtggtgcaatctcggctcaccacaccctccgcttcctgggttcaagcgattctcctgcctcagcctcccgagtagatgggattacaggcgcccaccaccacgcccggctattttttgtatttttagtagagatgaggtttcaccatgttggccaggctggtctcgaactcctgacctcaggtgatccacccacctcagcctcccaaagtgctgggattacaggtgtgagccaccacacccagccaatcatttgtatttctataccgtagcaacagacaatttgaaaatgaaatcttaaaaacaattccattgacagtagcatcaaataacataaaatagttcagaatagccaggtgtgatggcccgtgcctgtagtcccagctactctggatggctgaggtaggaggattgcttgagtccaggtggatcccttgagcccaagagttcaaggctgtagcacactataattgtgcctgtgaatagccactgcactccagcttgggcaacatagtgagactccgtctgtaaaataaaatgaaacaaaactaaaatactgctgagagaaattatagacctaagtaaatgaaaaggcataccatgcttatggattagaagactcaatattgttaagatgtcacttctccccaagtttatctgtaggttcaatgcaatcttgatcaaaatccccataaactttttttgggggaaggaaggggtaattgacaagttgattcttatttcatatggaaatgcacagggattcaaggttgcagtgacccctgtttgcaccactgcactccagcctgggagacagagtgagaccttgtctcaaaaaagaaattaaaaaaaaaaaaaaaaaaggaatgcaaagaatctcaaatagctaatatcttcacagtgaaaaacaaatttggagaacttagtagtacttgattttgagacttagttgaaatctaaagtaacctagagacaatggtattgtcaaagtagtaggaagtagaatataacaagagtcagaaatagacacatatacattcaatggacttggtttttttacaaaggtcccaaggctatccaatgccaaaaggaaaagttttcaacaaacggtgctgaaacaattagaaatatggaaaaagtgaacctggaccccccatctcacgccacaacaaaattattttgagatagtaatagacccagaagtaaaggctaaaaatataaatctaaaaggaaacaatattttcatgaccttggggtaggcaaagatttattaaagaggacacaaaaaacaacaaacagaagaaaaacctgaaaattgcatcaatataagtgtctgctcctcaaaagacacctttaagaaaatgaaatggtaagccacagactggaagaagatatttcgaatacatatatttaacaattttgtatcctgaataaataactcctgtaaatccataaaccaaaaaactccaaagtcaggcaaaagattggagcagatgcttcacaaaagatatatgaatgacccagtaagcacatgcaaatgtgcccaccattaattatatgggaaattaaaattaaaaccacaataatataccatttcacaccgaccaagatggaagttaaaattaaaaaaaaaaaagaaaggctgaccaatgtgtctatctactggagctctcctactgcgggtgggaatgtaaactggatgtaatcagtcattttggacaacaggtggcggtttcctataaagctaaacatagacttaccctggcacccagccgacgtgggagaactgggtacacaggtgtacactttacaaaactcatcgaattgcacactcagattgtgtgcattttactgtgtctaaacgttagcgctataaaagcaaaacaaaatataagatcataaaaataaaagcaattcctaggggtcctctccgttcccgggcttcctcggtgcggagggaaacgaaactagcccgaggcaaaacagcctccgcgaaccccgcccgccgctagttgcactttcgattttccctttag

SEQ ID NO: 2 - DNA, 24 bp, Artificial Sequence
  sense primer sequence
gtctatcacgaacaccatatgcca

SEQ ID NO: 3 - DNA, 25 bp, Artificial Sequence
  antisense primer sequence
ctaaagggaaaatcgaaagtgcaac

(19) United States
(12) Patent Application Publication
(10) Pub. No.: US 2008/0108067 A1
(43) Pub. Date: May 8, 2008

(54) Cloning of the retinoic acid inducible gene-1 promoter and uses thereof

(76) Inventors: Paul B. Fisher, Richmond, VA (US); Zhao-zhong Su, Richmond, VA (US)

Correspondence Address: BAKER BOTTS L.L.P., 30 ROCKEFELLER PLAZA, 44TH FLOOR, NEW YORK, NY, 10112-4498, US

(21) Appl. No.: 11/788,093

(22) Filed: Apr. 19, 2007

Related U.S. Application Data
(63) Continuation of application No. PCT/US05/37950, filed on Oct. 19, 2005.
(60) Provisional application No. 60/621,299, filed on Oct. 22, 2004.

(51) Int. Cl. C12Q 1/68; C12N 15/11; C12N 15/00; C07H 21/04; C12N 5/06; A01K 67/00

(52) U.S. Cl. 435/6; 536/24.1; 435/320.1; 435/325; 800/13; 536/25.3

(57) ABSTRACT

The present invention relates to the human Retinoic Acid Inducible Gene-1 (hereafter, “RIG-1”) promoter. The present invention provides for the promoter itself which is inducible by interferon, virus infection, retinoic acid and double-stranded RNA. A further embodiment includes expression constructs comprising the RIG-1 promoter operatively linked to a gene of interest, which may be a reporter gene or a therapeutic gene. It also provides for cells and non-human transgenic animals comprising such expression constructs. In addition, the present invention provides for methods of screening agents for anti-viral activity using RIG-1 promoter activation or inhibition as the basis of the screening system.

### GRANT INFORMATION

The subject matter of this provisional application was developed at least in part using funds from National Institutes of Health (National Institute of General Medical Sciences) Grant No. GM068448, principal investigator Dr. Paul B. Fisher, so that the United States Government has certain rights herein.

### INTRODUCTION

The present invention relates, at least in part, to the cloning of the human Retinoic Acid Inducible Gene 1 Promoter, and to the discovery that this promoter is inducible by interferon, virus infection, retinoic acid and double stranded RNA.

### BACKGROUND OF THE INVENTION

Under selective evolutionary pressure, host organisms have evolved anti-viral defense mechanisms. At the tissue and cellular level, recognition of the virally infected state is brought about by detection of viral products within and in the vicinity of infected cells. The Type-I interferons (IFNs) comprising IFN-α and IFN-β play a central role in anti-viral host defenses (De Clercq, 2004 Nat Rev Microbiol. 2:704-720; Li Y, et al., 2004 Arch Immunol Ther Exp (Warsz). IFN production initially occurs in infected cells and is then transferred to surrounding tissue or cells by secretion. Exposure of cells to IFNs results in the induction of an array of genes that are responsible for triggering various cellular pathways involved in protecting the host. IFNs are themselves induced by genes that sense viral infection and trigger the IFN pathway. Often, an IFN response may be sustained through a positive feedback loop where exposure of an IFN producing cell to more IFN or another inducer triggers a greater amount of production and secretion of IFN (Li Y, et al., 2004 Arch Immunol Ther Exp (Warsz). 52:156-163; Civas et al., 2002 Biochimie. 84:643-654; Brierley and Fish. J 2002 Interferon Cytokine Res. 22:835-845).

RNA helicases of the DEAD box and related DExD/H proteins form a very large superfamily of proteins that are evolutionarily conserved from bacteria and viruses to humans. DEAD box and related DExD/H helicases have seven to eight conserved motifs, the characteristics of which are used to subgroup members into individual families (Rocak and Linder, 2004 Nat Rev Mol Cell Biol 5:232-241; Lorsch, 2002 Cell. 28;109:797-800; Tanner and Linder, 2001 Mol Cell 2:251-262). They are associated with all processes involving RNA molecules, including transcription, editing, splicing, ribosome biogenesis, RNA export, translation, RNA turnover including via the RNAi mechanism, and organelle gene expression. A particular function of a helicase may predominate depending on the cellular milieu (Rocak and Linder, 2004 Nat Rev Mol Cell Biol 5:232-241; Lorsch, 2002 Cell. 28;109:797-800; Tanner and Linder, 2001 Mol Cell 2:251-262).

A commonly encountered by-product of viral replication is the intracellular presence of double-stranded RNA (dsRNA). The DExD/H box sub-family of RNA-helicases can act as transducers of anti-viral signals to trigger as well as sustain an antiviral response by recognizing and binding to dsRNA (Yoneyama et al., 2004 Nat Immunol. 5:730-737). The biological context of expression and function of four structurally related DExD/H RNA-helicases including human melanoma differentiation associated gene-5 (mda-5), human Retinoic Acid Inducible Gene-1 (RIG-1), porcine RHIV-1, and plant Q9SP32 are strikingly similar. RIG1 is expressed during all-trans retinoic acid-induced promyelocytic differentiation (Sun,1997 Ph.D. thesis Sanghai Second Medical University, Sanghai, China). It serves an essential function in dsRNA-induced innate antiviral responses (Yoneyama et al., 2004 Nat Immunol. 5:730-737) and is also induced by IFN-β in HO-1 melanoma cells (Kang et al., 2002 Proc Natl Acad Sci USA 99:637-642). The expression of RHIV-1 is induced by viral infection suggesting IFN inducibility (Zhang et al., 2000 Microb. Pathog. 28: 267-278). Q9SP32 (also known as CAF) seems to suppress cell division in floral meristems (Jacobsen et al., 1999 Development (Cambridge, U.K.) 126:5231-5243). The mda-5 gene was originally isolated as a sequence expressed during cell differentiation induced by IFN-β plus mezerein (MEZ), which involves growth inhibition (Kang et al., 2002 Proc Natl Acad Sci USA 99:637-642). Actual and putative helicases of this subgroup may participate in similar biochemical changes associated with growth inhibition, differentiation and anti-viral responses. Despite structural and functional homology, these and other related helicase molecules also have distinct biological roles (Rocak and Linder, 2004 Nat Rev Mol Cell Biol 5:232-241; Lorsch, 2002 Cell. 28;109:797-800; Tanner and Linder, 2001 Mol Cell 2:251-262).

The mda-5 RNA helicase contains a Caspase Recruitment Domain (CARD) in its Amino-terminal region and a DExH/D RNA helicase domain in its Carboxy-terminal region (Kang et al., 2002 Proc Natl Acad Sci USA 99:637-642). Data indicates that mda-5, by its ATP-dependent unwinding of RNA, may promote mRNA degradation and/or inhibit translation during growth inhibition and/or apoptosis mediated by IFN or TNF-α treatment (Kang et al., 2002 Proc Natl Acad Sci USA 99:637-642). The mda-5 gene is highly inducible by IFNs (especially IFN-β) regardless of cell type. There is relatively low basal expression level of mda-5 in various organs. The rapid induction of mda-5 by IFN treatment supports a critical role of mda-5 in responses that are specific for IFN signaling such as antiviral effects, growth inhibition, and apoptosis (Kang et al., 2002 Proc Natl Acad Sci USA 99:637-642).

The RIG-1 RNA helicase has two copies of a CARD domain in its Amino-terminal region and it contains a Carboxy-terminal helicase domain. The RNA helicases mda-5 and RIG-1 show 23% identity in their CARD and 35% identity in their helicase domains. Currently, both genes are more closely related to each other than to other known IFN-inducible RNA helicases (Kang et al., 2002 Proc Natl Acad Sci USA 99:637-642; Yoneyama et al., 2004 Nat Immunol. 5:730-737). RIG-1 differs from mda-5 in having two CARD domains. The CARD domain in both molecules is presumed to provide protein-interaction domains, providing specific docking sites or interfaces between molecules that transmit or transduce signals involved in regulating programmed cell death (Kang et al., 2002 Proc Natl Acad Sci USA 99:637-642; Yoneyama et al., 2004 Nat Immunol. 5:730-737). Since RIG-1 differs from other helicases, including mda-5, in having a tandemly repeated CARD motif, its range of molecular interaction is likely to be different from mda-5 and other related helicases. Studies on the effect of RIG-1 expression utilizing a RNA interference (RNAi) strategy have been performed. This strategy determines the cellular effect of ablating expression of a specific gene in a cell population, RIG-1 in this case. Impairment of RIG-1 expression resulted in impaired activation of IFNs following challenge by viral infection (Yoneyama et al., 2004 Nat Immunol. 5:730-737). Experiments have been also been performed to determine the functional significance of the RIG-1 CARD domain. Expression of the CARD motif alone was able to activate RIG-1 transduced downstream signals (Yoneyama et al., 2004 Nat Immunol. 5:730-737). In contrast, RIG-1 molecules mutated to disrupt the CARD region did not activate known target downstream genes (Yoneyama et al., 2004 Nat Immunol. 5:730-737). The end result of having reduced RIG-1 activity was an impaired IFN response following treatment with inducers. The target specificity of CARD domains has been demonstrated wherein the CARD domain of RIG-1 was seen to activate two distinct regulators, NF-κB and IRF-3. CARD domains derived from Nod-1 and Nod-2 (unrelated CARD containing proteins) only activated NF-κB but not IRF-3 (Yoneyama et al., 2004 Nat Immunol. 5:730-737). Evidence has been presented showing that mere overexpression of RIG-1 in cells does not induce IFNs. However, once cells are treated with an inducer such as dsRNA, IFN production rapidly occurs. This demonstrates regulation of the RIG-1 protein and requirement for the presence of an activator such as dsRNA to convert it into an active molecule (Yoneyama et al., 2004 Nat Immunol. 5:730-737).

### SUMMARY OF THE INVENTION

The present invention relates to the human Retinoic Acid Inducible Gene-1 (hereafter, “RIG-1”) promoter. It is based on the cloning of this promoter and on the discovery that the promoter is inducible by interferon, virus infection, retinoic acid and double-stranded RNA.

The present invention provides for the promoter itself, as well as expression constructs comprising the RIG-1 promoter operatively linked to a gene of interest, which may be a reporter gene or a therapeutic gene (e.g., an anti-viral agent or an anti-proliferative agent other than RIG-1 itself). It also provides for cells and non-human transgenic animals comprising such expression constructs.

In addition, the present invention provides for methods of screening agents for anti-viral activity or anti-proliferative therapy using the RIG-1 promoter. The ability of a test agent to induce the RIG-1 promoter is consistent with anti-viral activity or anti-proliferative activity.

In addition, the present invention provides for methods of screening agents for therapy of non-viral diseases or disorders using the RIG-1 promoter. The ability of a test agent to inhibit activity of the RIG-1 promoter is consistent with therapeutic activity in cases where activation of the associated gene pathway is pathogenic or cancer inducing.

### BRIEF DESCRIPTION OF THE FIGURES

FIG. 1A-C. Sequence of the human RIG-1 promoter.

FIG. 2. Activity of the RIG-PROM-LUC reporter in response to inducers in immortalized normal melanocytes, immortalized normal astrocytes, breast and cervical cancer cells.

FIG. 3. Activity of the RIG-PROM-LUC reporter in response to viral infection.

FIG. 4. Activity of the RIG-PROM-LUC reporter in response to inducers in immortalized normal prostate epithelial cells, prostate cancer, melanoma and glioblastoma cells.

FIG. 5A-C. Activity of the RIG-PROM-LUC reporter in response to inducers in immortalized normal prostate epithelial, human prostate and cervical cancer cells.

### DETAILED DESCRIPTION OF THE INVENTION

For clarity, and not by way of limitation, the detailed description of the invention is divided into the following subsections:
(i) the RIG-1 promoter:
(ii) RIG-1 promoter expression constructs;
(iii) assay systems using RIG-1 promoter expression constructs; and
(iv) therapeutic uses of RIG-1 promoter expression constructs.

### The RIG-1 Promoter

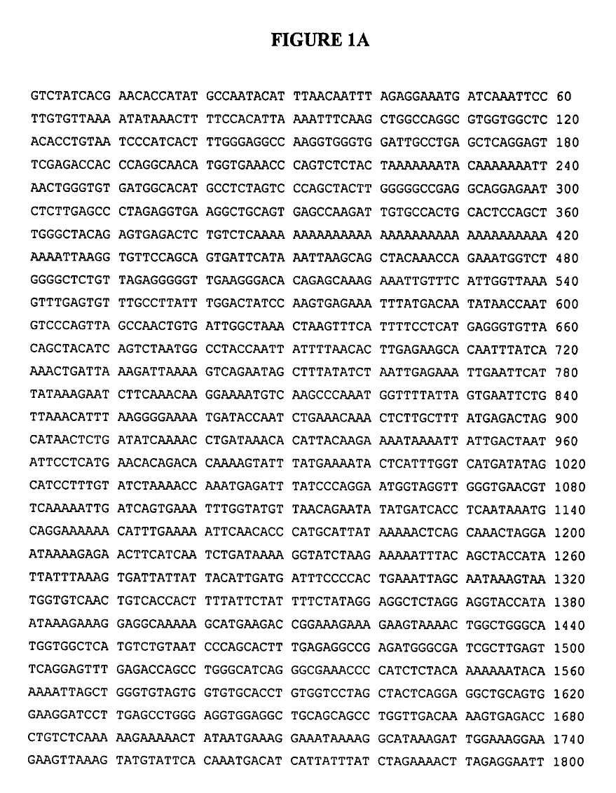

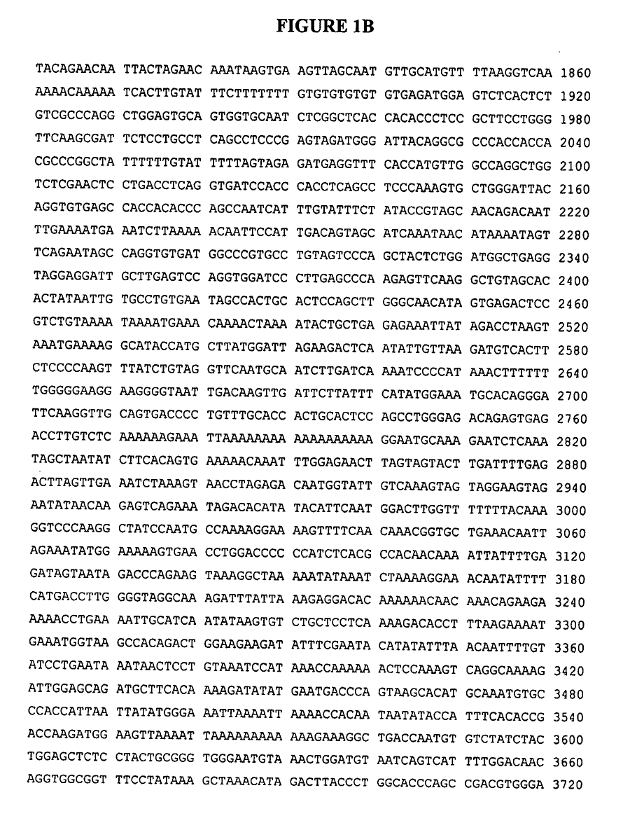

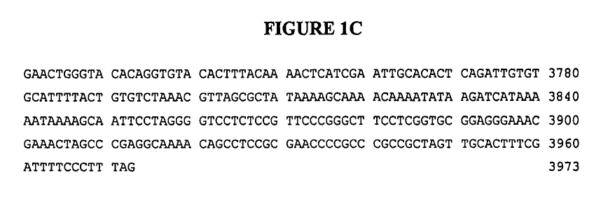

In a non-limiting specific embodiment, the present invention provides for a RIG-1 promoter comprising (SEQ ID. NO:1, FIG. 1), as contained in plasmid RIG-PROM-LUC, deposited with the American Type Culture Collection, Patent Depository, 10801 University Blvd., Manassas, Va. 20110 on Oct. 11, 2004 and assigned Accession No. PTA-6251.

In further non-limiting embodiments, the present invention provides for a RIG-1 promoter, which hybridizes to the Retinoic Acid Inducible Gene-1 promoter having SEQ ID NO:1 or of plasmid RIG-PROM-LUC under stringent hybridization conditions, for example hybridization in 0.5 M NaHPO₄, 7 percent sodium dodecyl sulfate (“SDS”), 1 mM ethylenediamine tetraacetic acid (“EDTA”) at 65° C., and washing in 0.1×SSC/O.0.1 percent SDS at 68° C. (Ausubel et al., 1988, Current Protocols in Molecular Biology, Vol. I, Green Publishing Associates, Inc., and John Wiley and Sons, Inc. New York, at p.2.10.3).

In still further embodiments, the present invention provides for a RIG-1 promoter, obtained by using primers (SEQ ID NO:2 and SEQ ID NO:3) in a polymerase chain reaction (PCR) with human genomic DNA as template. Isolated and purified genomic DNA is subjected to 37 cycles of PCR as follows: Temperature-94° C., cycle time-2 min for 1 cycle; Temperature-94° C., cycle time-1 min, Temperature-60° C., cycle time-2 min, Temperature-72° C. cycle time-4 min for 35 cycles; Temperature-72° C., cycle time-10 min for 1 cycle. The present invention encompasses RIG-1 promoters which hybridize, under stringent hybridization conditions (supra) to the promoter generated by the foregoing PCR method.

In a further embodiment, the invention provides for RIG-1 promoters at least 80,85, 90 or 95% homologous to the RIG-1 promoter sequence (SEQ ID NO:1) as determined by computer homology algorithms and associated software including but not limited to BLAST, FASTA etc.

A RIG-1 promoter of the invention is inducible by interferon, viral infection, retinoic acid and/or double-stranded RNA. In specific non-limiting embodiments, the RIG-1 promoter, contained in a cell, is inducible by exposing the cell to α or β-interferon at a concentration of 10-2000 units/ml, and/or vesicular stomatitis virus (VSV) at a multiplicity of infection (moi) of 1, all-Trans-retinoic acid at a concentration of 10 μM and/or double stranded RNA such as poly IC:poly IC at a concentration of 10 μg/ml. _

### RIG-1 Promoter Expression Constructs

The present invention provides for a nucleic acid comprising a RIG-1 remoter as described in the preceding section operably linked to a gene of interest, where the gene of interest may be, but more preferably is not the Retinoic Acid Inducible Gene-1. The gene of interest may be, for example and not by way of limitation, a reporter gene or a therapeutic gene.

Suitable reporter genes include, but are not limited to Chloramphenicol Acetyl-Transferase (CAT) (Boshart et al., 1992 Gene 110:129-130), Luciferase (Sherf and Wood 1992 Promega Notes 49:14-21), Secreted Alkaline Phosphatase (SEAP) (Berger et al., 1988 Gene. 66:1-10), Green, Cyan, Yellow, Red and Far Red Fluorescent Proteins (Kain et al., 1995 Biotechniques 19:650-655, Living Colors Users Manual (2001), BD Life Sciences, Palo Alto, Calif.), and β-Galactosidase (Lim and Chae, 1989 Biotechniques 7:576-579).

Suitable therapeutic genes include, but are not limited to β-Interferon, Protein Kinase R (PKR), melanoma differentiation associated gene-5 (mda-5), melanoma differentiation associated gene-7 (mda-7), p21^{WAF1,CIP1}, pRb or p53 or antisense oriented genes encoding k-RAS, H-RAS, c-myc, BCL-2, BCL_{XL} or E2F-1.

Such nucleic acids may be comprised in vectors, which may be in specific embodiments a replicable vector, an expression vector or a gene transfer vector. As non-limiting examples, vectors may include plasmid vectors, cosmid vectors, bacteriophage vectors, adenoviral vectors, retroviral vectors, lentiviral vectors or other virus-based vectors well-known to ordinary skilled practitioners.

Examples of appropriate virus-based gene transfer vectors include, but are not limited to, those derived from retroviruses, for example Moloney murine leukemia-virus based vectors (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96:22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

Suitable expression vectors include, but are not limited to constructs comprising the RIG-1 promoter operatively linked to a gene of interest in combination with additional genes or gene-expression regulating elements as described herein. Such vectors may comprise further elements which confer properties such as antibiotic resistance including but not limited to puromycin, neomycin, hygromycin and zeocin. Additionally such vectors may comprise an Internal Ribosome Entry Site (IRES) to enable co-expression of two or more genes as multicistronic messages. Additionally, such vectors may comprise polyadenylation sites derived from the Bovine Growth Hormone gene (BGH), the herpes virus Thymidylate Kinase gene (tk) or other natural or synthetic polyadenylation sites known to those skilled in the art. As an example to obtain these vectors, insert and vector DNA can both be exposed to a restriction enzyme to create complementary ends on both molecules which base pair with each other and are then ligated together with DNA ligase. Alternatively, linkers can be ligated to the insert DNA which corresponds to a restriction site in the vector DNA, which is then digested with the restriction enzyme which cuts at that site. Other means are also available and known to an ordinary skilled practitioner.

In preferred, non-limiting embodiments of the invention, the expression vector is an E1 -deleted human adenovirus vector of serotype 5. To prepare such a vector, an expression cassette comprising the RIG-1 promoter element operatively linked to a gene of interest and a polyadenylation signal sequence may be inserted into the multiple cloning region of an adenovirus vector shuttle plasmid, for example a promoterless shuttle vector pZeroTg. In the context of this plasmid, the expression cassette may be inserted into the DNA sequence homologous to the 5′ end of the genome of the human serotype 5 adenovirus, disrupting the adenovirus E1 gene region. Transfection of this shuttle plasmid into the E1-transcomplementing 293 cell line (Graham et al., 1977, J. General Virology 36:59-74), or another suitable cell line known in the art, in combination with either an adenovirus vector helper plasmid such as pJM17 (Berkner, 1988, Biotechniques 6:616-624; McGrory et al., 1988, Virology 163:614-617) or pBHG10 (Bett et al., 1994, Proc. Natl. Acad. Sci. U.S.A. 91:8802-8806) or a Clal-digested fragment isolated from the adenovirus 5 genome (Berkner, 1988, Biotechniques 6:616-624), allows recombination to occur between homologous adenovirus sequences contained in the adenovirus shuttle plasmid and either the helper plasmid or the adenovirus genomic fragment. This recombination event gives rise to a recombinant adenovirus genome in which the cassette for the expression of the foreign gene has been inserted in place of a functional E1 gene. When transcomplemented by the protein products of the human adenovirus type 5 E1 gene (for example, as expressed in 293 cells), these recombinant adenovirus vector genomes can replicate and be packaged into fully-infectious adenovirus particles. The recombinant vector can then be isolated from contaminating virus particles by one or more rounds of plaque purification (Berkner, 1988, Biotechniques 6:616-624), and the vector can be further purified and concentrated by density ultracentrifugation. In a non-limiting embodiment, the adenovirus vector is modified to enhance infectivity by modification of viral capsid components or creation of chimeric infectivity enhanced adenovirus vectors for gene delivery (Glasgow et al., Virology. 2004, 324:103-116).

A vector or an expression vector may be comprised in a cell. Various mammalian cells may be used as hosts, including, but not limited to, the mouse fibroblast cell NIH3T3, CHO cells, HeLa cells, Ltk.sup.-cells, Cos cells, HO-1 human melanoma cells, immortalized human primary astrocytes, immortalized normal human melanocytes, immortalized human prostate epithelial cells or various primary mammalian cells known to a skilled practitioner.

Nucleic acid comprising a RIG-1 promoter operably linked to a gene of interest may be used to transfect mammalian cells by methods well-known in the art such as calcium phosphate precipitation, lipofection, electroporation or may be introduced by other methods such as microinjection, bioballistics etc.

A nucleic acid comprising a RIG-1 promoter expression construct may constitute a transgene in a non-human transgenic animal, such as but not limited to a mouse, a rat, a sheep, a goat, a rabbit or a pig.

### Assay Systems Using RIG-1 Promoter Expression Constructs

The present invention provides for a method for determining whether a test agent modulates the activity of the RIG-1 promoter and/or has therapeutic (e.g. antiviral or anti-proliferative) activity, comprising:
(i) providing a cell containing a nucleic acid comprising a RIG-1 promoter operably linked to a reporter gene;
(ii) exposing the cell to the test agent; and
(iii) determining whether exposure to the test agent results in an increase in expression of the reporter gene; or
(iii) determining whether exposure to the test agent results in a decrease in expression of the reporter gene;
   wherein an increase in the expression of the reporter gene indicates that the test agent has therapeutic activity and a decrease indicates that the test agent is an antagonist of IFN, retinoic acid and dsRNA induced signaling.

A “test agent” may be, for example but not by way of limitation, a comound that acts as an agonist or an antagonist of IFN-β, IFN-α or IFN-γ. In one embodiment, the compound is a small organic molecule having a weight of about 5 kilodaltons or less that acts as an agonist or an antagonist of IFN-β, IFN-α or IFN-γ. In another embodiment, the compound is a molecule that induces the production of IFN-β, IFN-α or IFN-γ in a cell. In another embodiment the compound is a small organic molecule having a weight of about 5 kilodaltons or less that acts as an agonist or an antagonist of IFN-β, IFN-α or IFN-γ production in a cell.

In a non-limiting embodiment, the test agent by functioning as an antagonist of IFN-β, IFN-α or IFN-γ signaling in a cell, could be therapeutically useful in diseases involving but not limited to activation of the STAT genes (Calo et al., J Cell Physiol. 2003 197:157-168).

Further, a “test agent” may be, for example but not by way of limitation, a compound that acts as an agonist or an antagonist of retinoic acid signaling. In one embodiment, the compound is a small organic molecule having a weight of about 5 kilodaltons or less that acts as an agonist or an antagonist of retinoic acid signaling.

In various embodiments a “test agent” may be, for example but not by way of limitation, a compound that acts as an agonist or an antagonist of double stranded RNA induced signaling. In one embodiment, the compound is a small organic molecule having a weight of about 5 kilodaltons or less that acts as an agonist or an antagonist of double stranded signaling.

A test agent in non-limiting embodiments may be a member of a library or family of compounds generated by combinatorial chemistry, may be an existing compound, or may be rationally designed.

In specific non-limiting embodiments, the invention provides for assay systems that utilize a recombinant expression construct effective in directing the transcription of a selected coding sequence which comprises (a) a RIG-1 promoter (SEQ ID NO:1); and (b) a coding sequence operably linked to the promoter, whereby the coding sequence can be transcribed and translated in a host cell and the promoter is heterologous to the coding sequence. In non-limiting embodiments, the coding sequence is chosen from a list of “reporter genes” including but not limited to Luciferase, Green, Cyan, Yellow, Red or Far Red Fluorescent Protein, Secreted Alkaline Phosphatase and β-Galactosidase.

In a specific non-limiting embodiment, the RIG-1 promoter (SEQ ID NO:1) is cloned into the pGL3 luciferase reporter system (Promega Inc. Madison, Wis.) and designated RIG-PROM-LUC deposited with the American Type Culture Collection, Patent Depository, 10801 University Blvd., Manassas, Va. 20110 on Oct. 11, 2004 and assigned Accession No. PTA-6251.

The invention further provides for an assay system utilizing the recombinant expression construct (supra) “introduced” into a host cell. The recombinant expression construct may be introduced either in a (a) transient manner, wherein re-introduction of the recombinant expression construct into the host cell may be necessary for each new cycle of testing or (b) a stable manner into the host cell, wherein the recombinant expression construct is stably integrated into the host cell DNA.

In various embodiments of the invention, the expression construct may be introduced by means selected from a group comprising but not limited to, liposome mediated transfer, microinjection, electroporation, adenovirus infection, retrovirus infection or lentivirus infection.

The invention further provides for an assay system comprising (i) a recombinant expression construct operably linked to a reporter gene or a gene whose activity modulates the growth of a cells (ii) introducing the construct into a host cell system (iii) exposing the cell to a test agent (iv) measuring the amount of reporter gene product produced by the cell or measuring growth rate of the exposed cell (v) comparing the amount of reporter gene product or effect on growth rate of a cell exposed to the test agent to the amount of reporter gene product or effect on growth rate of a cell not exposed to the test agent. To provide an adequate control, the cell (generally in a cell culture or organism) exposed to the test agent and cell not exposed to the test agent should otherwise be maintained under the same or similar conditions. Contact with the test agent is for an effective time followed by measurement of activity or effect o growth of the gene of interest operably linked to the RIG-1 promoter in the recombinant expression construct. An “effective” time is a period of time sufficient to induce activity of the RIG-1 promoter by at least 30 percent. The measurement of reporter gene activity or effect on cell growth is performed in accordance with the type of reporter gene system or growth assays utilized as known to an ordinary skilled practitioner.

In a particular embodiment, it may be desirable to boost the activity of the RIG-1 promoter using an activation agent. The present invention provides for an assay system wherein the activity of the RIG-1 promoter is activated prior to exposure of the assay system to a test agent. Activation agents include but are not limited to α or β-interferon at a concentration of 10-2000 units/ml, and/or vesicular stomatitis virus (VSV) at a multiplicity of infection (moi) of 1, all-Trans-retinoic acid at a concentration of 10 μM and/or double stranded RNA such as poly IC:poly IC at a concentration of 10 μg/ml.

In related embodiments, the present invention provides for kits for practicing such assay methods. For example, but not by way of limitation, the invention provides for a kit containing a nucleic acid comprising a RIG-1 promoter operably linked to a reporter gene, a reporter gene detecting agent and associated reagents.

As a specific non-limiting embodiment, a kit utilizing the RIG-1 promoter provides for a RIG-PROM-LUC luciferase test-kit system. It further comprises of reagents to introduce the promoter into a host cell including but not limited to reagents required for lipofection or an adenovirus or retrovirus vector harboring the RIG-PROM-LUC. The kit may in a further embodiment provide a cell line in which the reporter system is stably integrated. The kit may further comprise an activator, a cell lysis buffer and luciferin/ATP mixture to measure the activity of the luciferase reporter. Utilization of a kit comprising the various components (supra) will allow measurement of reporter activity and provide an assay system for measuring the antagonistic or agonist activity of a test compound.

### Therapeutic Uses of RIG-1 Promoter Expression Constructs

The present invention provides for methods of protecting against or limiting viral infection in a subject comprising administering, to the subject, an agent which increases the level of an anti-viral therapeutic gene by increasing the activity of a RIG-1 promoter “anti-viral therapeutic gene construct” in a subject. “Protecting against a viral infection” does not require that there be absolute protection; for example, substantially decreasing the likelihood of infection, or for example reducing the risk of infection, with exposure, at least 2-fold, is considered protection. “Limiting viral infection”, as defined herein, refers to any one or more of decreasing the number of cells infected by a virus within a subject or among several subjects; decreasing the cytopathic effect caused by a virus, decreasing the amount of viral replication, decreasing the clinical severity of a viral infection, and/or decreasing the period of time that a subject suffers from a viral infection. “Increasing the level of an anti-viral therapeutic gene in the subject” encompasses increasing the level of therapeutic protein activity or other gene product such as antisense RNA in at least some but not necessarily all cells, tissues, and/or other body fluids of the subject. The level of anti-viral gene derived activity may be measured directly or indirectly, for example, but not by way of limitation, by measuring the amount of RNA encoding the gene, the amount of protein product expressed or effective reduction of viral titer in tissue, blood or other body fluid of the treated subject.

In further, non-limiting embodiments, the present invention provides for a method of protecting against or limiting viral infection in a subject comprising administering, to the subject, a therapeutically effective amount of IFNs α-, β-, γ-, retinoic acid or double stranded RNA which increases RIG-1 promoter activity in the subject.

As non-limiting examples, consider the following:

A therapeutic amount of RIG-1 promoter expression construct may be administered to a subject by any suitable route, including intravenously, intramuscularly, orally, or by inhalation, so as to introduce the RIG-1 promoter expression construct into cells which are infected or at risk of being infected. The construct may be comprised in a vehicle which aids cellular uptake and/or inhibits degradation, for example, in liposomes or microspheres. As a specific non-limiting embodiment, an effective amount of RIG-1 promoter expression construct may be administered to a subject who has been or may be in contact with a virus spread by the respiratory route in the form of an inhaled aerosol spray.

A RIG-1 promoter expression construct nucleic acid may be administered in expressible form, for example, in the form of “naked DNA” or comprised in a vector, for example a viral vector. In particular non-limiting embodiments the virus may be an adenovirus, such as, for example, a replication-defective adenovirus. As a specific non-limiting embodiment, an effective amount of a RIG-1 promoter construct, comprised in a viral vector such as a replication defective adenovirus vector, may be administered to a subject who has been or may be in contact with a virus spread by the respiratory route in the form of an inhaled aerosol spray. For example, the amount of virus administered may be 10⁹-10¹³ plaque forming units. In a further non-limiting embodiment, the adenovirus vector is modified to enhance infectivity by modification of viral capsid components or creation of chimeric infectivity enhanced adenovirus vectors for gene delivery and administered to a subject at a amount ranging from 10⁹ to 10¹³ plaque forming units (Glasgow et al., Virology. 2004, 324:103-116).

In further embodiments of the invention, an effective amount of a second agent may be administered to the subject before, during, or after administration of RIG-1 promoter construct. The second agent is defined as a facilitator of RIG-1 promoter activity. Examples include, but are not limited to, Interferon α-, β-, γ-, retinoic acid or double stranded RNA, or a test agent identified as increasing RIG-1 promoter activity. In additional embodiments, a third agent which promotes RIG-1 promoter activity may be additionally administered; for example, but not by way of limitation, if one of the above interferons is used as second agent, another one of these interferons may be used as third agent.

In still luther embodiments of the invention, whether or not a second and/or third agent is used, an effective amount of an antiviral agent is administered to improve the net antiviral defense. The amount of antiviral activity achieved is greater that the amount associated with the antiviral agent used without a RIG-1 promoter construct. Examples of antiviral agents include, but are not limited to, (i) amantadine, rimantidine, and sialic acid analogues such as zanamivir or other inhibitors of influenza sialidase for the specific inhibition of influenza viruses; (ii) Aayclovir (Zovirax), ganciclovir, valacyclovir (Valtrex), lamivudine (3TC; Epivir) or other nucleoside analogs such as vidarabine for the treatment of herpesvirus infections (HSV-1, HSV-2, VZV (chicken pox), EBV and CMV); (iii) foscarnet (Foscavir), a pyrophosphate analog, may be useful in the treatment of the above-mentioned herpesviruses, and also may useful in the treatment of retroviruses including HIV-1; (iv) zidovudine (Retrovir or AZT), didanosine, zalcitabine and related purine analogues such as ribavirin, which are inhibitors of viral reverse transcriptase, for the treatment of retroviruses including HIV-1 (ribavirin is also useful in the treatment of infections caused by arenaviruses (e.g. Lassa fever) or bunyaviruses (e.g. Hantavirus), respiratory syncytial virus (RSV) and in the treatment of Hepatitis C; the non-nucleoside reverse transcriptase inhibitor (NNRTI) nevirapine (Viramune) may also be useful in the treatment of HIV-1); (v) indinavir (Crixivan), saquinavir (Fortovase) and other HIV protease inhibitors for the treatment of retroviruses including HIV-1; (vi) monoclonal antibody inhibitors of ICAM-1 for the treatment of rhinoviruses, the most common cause of colds; and (vii) zintevir, an HIV integrase inhibitor, for the treatment of HIV-1.

Many of the above-mentioned drugs can be used in combination to achieve anti-viral effects that are synergistic over those achieved by single-drug therapy, although the choice of combinations must be judicious.

Examples of infections which may be treated by the foregoing methods are those caused by 1) picornaviruses, a family of viruses that includes polioviruses, Coxsackie viruses, rhinoviruses, enteroviruses, and hepatitis A virus, which can cause poliomyelitus, meningitis, and hepatitis (hepatitis A) in humans, 2) caliciviruses, a family of viruses that include the mild gastroeneteritis-inducing Norwalk group of viruses, 3) astroviruses, which can cause severe gastroenteritis, 4) togaviruses, a family of viruses that includes the alphaviruses, the cause of polyarthritis and a variety of encephalitidies including Eastern and Western equine encephalitis, and rubiviruses, the cause of rubella (German measles), 5) flaviviruses, a family of viruses including flaviviruses, pestiviruses and hepatitis C virus, which can cause St. Louis and West Nile encephalitis among others, tick borne encephalitis, Dengue fever, yellow fever, hepatitis (hepatitis C), and some types of hemorrhagic fevers, 6) coronaviruses, a family of viruses including the coronaviruses, a cause of colds in humans and feline infectious peritonitis in cats, and the toroviruses, 7) arteriviruses, 8) paramyxoviruses, a family of viruses including paramyxoviruses, rubulaviruses, morbilliviruses and pneumonoviruses, which can cause measles and mumps, 9) rhabdoviruses, a family of viruses including rhabdoviruses, vesciculoviruses, lyssaviruses, ephemeroviruses, cytorhabdoviruses and nucleorhabdoviruses, which can cause vescicular stomatitis and rabies among other diseases, 10) filoviruses, notorious as the cause of Ebola and related hemorrhagic fevers, 11) orthomyxoviruses, a family of viruses including influenza A, B and C viruses, major causative agents of influenza infections, 12) bunyaviruses, a family of viruses including bunyaviruses, phleboviruses, nairoviruses, hantaviruses and tospoviruses, viruses associated with the hemorrhagic Hanta fever, encephalitis, and Rift Valley fever, 13) arenaviridae, the cause of Lassa fever and hemorrhagic fevers such as Bolivian, Argentinian or Venezualan hemorrhagic fevers, 14) reoviruses, a family of viruses including orthoreoviruses, responsible for a host of respiratory and enteric infections, orbiviruses, responsible for Colorado tick fever, rotaviruses, a major cause of morbidity stemming from persistent diarrhea in the developing world, coltiviruses, aquareoviruses, cypoviruses, phytoreoviruses, fijiviruses and orzyaviruses, 15) birnaviruses, a family of viruses including aquabirnaviruses, avibirnaviruses and entomobirnaviruses, 16) retroviruses, a family of viruses containing lentiviruses such as HIV-1 and -2, the causative agent in AIDS, spumaviruses and a range of retroviruses including oncogenic viruses such as the leukemia-inducing HTLV family of viruses and type A, B, C or D retroviruses, 17) hepadnaviruses, a family of viruses including orthohepadnaviruses and avihepadnaviruses, the cause of hepatitis B, 18) circoviruses, 19) parvoviruses, a family of viruses including the chordoparvovirus and entomoparvovirus subfamily comprising erythroviruses, dependoviruses, entomoparvoviruses, densoviruses, iteraviruses and contraviruses, 20) papovaviruses, a family of viruses including papillomaviruses, the cause of warts and some cancers of the cervix and other gential regions, and polyomaviruses, the cause of progressive multifocal leukoencephalopathy and some human malignancies, 21) adenoviruses, a family of viruses including mastadenoviruses and aviadenoviruses, which can cause flu-like respiratory infections in humans, 22) herpesviruses, a family of viruses including the alphaherpesvirus, betaherpesvirus, and gammaherpesvirus subfamilies comprising simplexviruses, varicelloviruses, cytomegaloviruses, muromegaloviruses, roseoloviruses, lymphocryptoviruses and rhadinoviruses, the cause of chicken pox, recurring infections of the oral cavity or the genital tract, mononucleosis and some human malignancies including Burkitt's lymphoma and Hodgkin's disease, 23) poxviruses, a family of viruses including the chordopoxvirus and entemopoxvirus subfamilies comprising orthopoxviruses, the source of smallpox infection, parapoxviruses, avipoxviruses, capripoxviruses, lepropoxviruses, suipoxviruses, molluscipoxviruses, yatapoxviruses, and entomopoxviruses A, B and C, 23) unnamed viruses of the iridovirus family and those causing African Swine Fever, and 24) unclassified human and animal viruses including Borna Disease virus, hepatitis E and X viruses and unclassified arboviruses.

In further, non-limiting embodiments, the present invention provides for methods of inhibiting cell proliferation and/or promoting apoptosis of a cell population or in a subject comprising administering, to the cell population or subject, an agent which increases the level of anti-proliferative or apoptosis inducing gene activity in the subject. Inhibiting cell proliferation and/or promoting apoptosis means decreasing the increase in the number of cells in a population over a time interval, relative to a control population in which the level of RIG-1 promoter activity driving anti-proliferative or apoptosis inducing gene has not been increased over the same length of time, by at least about 20 percent. “Increasing the expression of RIG-1 promoter construct in the subject” encompasses increasing the level of RIG-1 promoter construct expressed gene product in at least some but not necessarily all cells, tissues, and/or fluids of the subject.

In related embodiments, the present invention provides for methods of inhibiting tumor growth in a subject comprising administering, to the subject, an effective amount of an agent which increases the expression of RIG-1 promoter construct in the subject.

Examples of agents which may be used to increase the expression of RIG-1 promoter construct activity have been described in the foregoing section.

In particular embodiments, it may be desirable to co-administer an effective amount of IFN α-, β-, γ-, retinoic acid or double stranded RNA which increases RIG-1 promoter activity in the subject.

The foregoing agents may be administered in conjunction with one or more additional anti-neoplastic agent, including, but not limited to, a chemotherapeutic agent, immunotherapy, radiation therapy, and the like.

Examples of malignancy which may be treated according to the present invention include, but are not limited to, melanoma, glioblastoma multiforme, neuroblastoma, astrocytoma, osteosarcoma, breast cancer, cervical cancer, colon cancer, lung cancer, Kaposi's sarcoma, hairy cell leukemia, nasopharyngeal cancer, ovarian cancer, and prostate cancer.

### Experimental Details

### Materials and Methods

Human cell lines, cell culture and DNA transfection: Human cervical carcinoma (HeLa), breast carcinoma (T47D) and prostate carcinoma (DU-145) derived cell lines were obtained from the ATCC (Manassas, Va.) and 70 W, p69, G18, , FM-516 were obtained from their originating laboratories. PHFA was isolated and characterized by the inventors. All cell lines except p69 were grown in Dulbecco's Modification of Eagle's Medium (DMEM) supplemented with 10% fetal calf serum and maintained in an cell culture incubator at 37° C. with 5% CO₂ atmosphere and 100% humidity. The p69 cell line was grown in RPMI medium supplemented with 10% fetal calf serum and maintained in an cell culture incubator at 37° C. with 5% CO₂ atmosphere and 100% humidity. Transfection to introduce plasmid DNA (the pGL3 vector or RIG-1 promoter cloned in the pGL3 vector RIG-PROM-LUC) into cells for gene expression was performed utilizing Lipofectamine 2000 reagent according to conditions recommended by the manufacturer (Invitrogen, Carlsbad, Calif.).

Construction of the RIG-1 promoter luciferase reporter and assaying luciferase activity: Normal human genomic DNA was isolated and purified from human fetal astrocyte cells. A PCR reaction was performed on 100 ng of genomic DNA using forward and reverse PCR primers SEQ ID NO:2 and SEQ ID NO:3. The PCR conditions were as follows: Temperature-94° C., cycle time-2 min for 1 cycle; Temperature-94° C., cycle time-1 min, Temperature-60° C., cycle time-2 min, Temperature-72° C. cycle time-4 min for 35 cycles; Temperature-72° C., cycle time-10 min for 1 cycle. A total of 37 cycles was performed and an approximately 4 kilobase DNA fragment was obtained. This fragment was cloned into the pGL3-Basic luciferase reporter plasmid using standard DNA ligation techniques. The restriction enzyme sites utilized to clone the DNA was 5′ HindIII and 3′ XhoI. This luciferase reporter construct was tested by transfecting plasmid DNA into cells, treating cells with inducers including β-interferon at a concentration of 100-2000 units/ml, vesicular stomatitis virus (VSV) at a moi of 1, all Trans-retinoic acid at a concentration of 10 μM and double stranded RNA such as poly IC:poly IC at a concentration of 10 μg/ml where applicable. Treatment with inducer was for 6 h followed by overnight incubation of cells and assaying for luciferase activity utilizing a luciferase assay kit from Promega Inc. (Madison, Wis.) according to instructions provided by the manufacturer.

### Results

FIG. 1A-C shows the sequence of the genomic DNA PCR amplified from human genomic DNA and cloned into the luciferase reporter (SEQ ID NO:1). The putative transcription start site is a “T-residue” at position 3971 of SEQ ID NO:1 and putative transcription factor binding sites include Ik-2, c-Ets, AP-1, C/EBPb, at positions 3615 to 3650 of SEQ ID NO:1. Putative IRF-2 and IRF-1 binding sites are present at positions 3931 to 3941 and 3950 to 3967 respectively of SEQ ID NO:1.

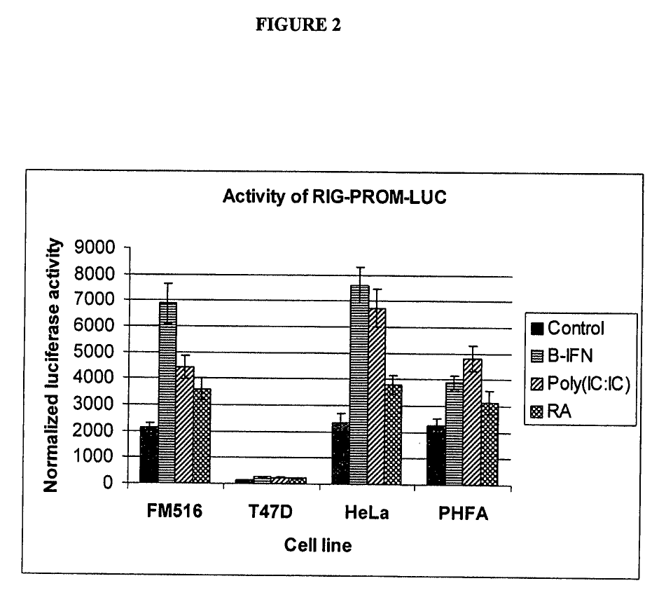

The reporter construct was tested for activity by transfecting it into FM516 immortalized normal human melanocytes, T47D breast carcinoma cells, HeLa cervical carcinoma cells, and immortalized primary human fetal actrocytes (PHFA). One set of cells (Control) was left untreated. Three additional transfected plates from each cell line was treated with either IFN-β), PolyIC:PolyIC (dsRNA) or retinoic acid (RA) as described in material and methods. FM516 and HeLa cells showed robust activation of promoter activity with IFN-β induction being greater than PolyIC:PolyIC and least with retinoic acid (FIG. 2). PHFA cells showed a lower overall by significant response to the inducers used while T47D cells showed extremely low activity or activation with the inducers. The reason for lack of activity in T47D is not currently known. However, it is apparent that the RIG-1 promoter sequence isolated by PCR is active and responds well to inducers in many cell types. Response to IFN-β ranged from 2 to 3.5-fold, to PolyIC:PolyIC from 2-3-fold and for RA from around 1.5 fold compared to untreated controls depending on the cell line (FIG. 1).

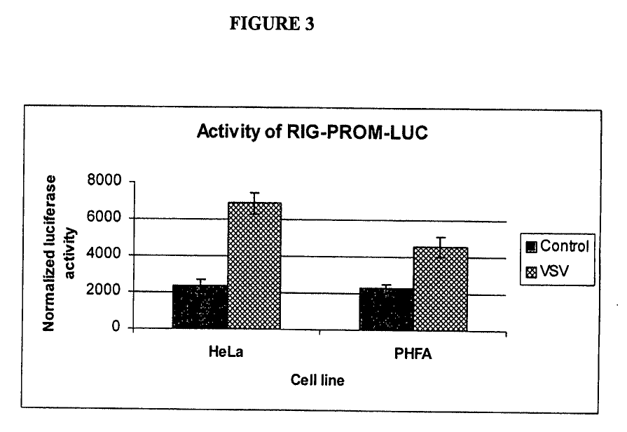

Similar tests of promoter activation were performed utilizing Vesicular Somatitis Virus as a potential inducer of RIG-1 promoter activity as measured by luciferase and in comparison to control uninfected cells (FIG. 3). These experiments show that the RIG-1 luciferase reporter is inducible following viral infection by approximately 3.5 and 2-fold respectively in HeLa and PHFA cells.

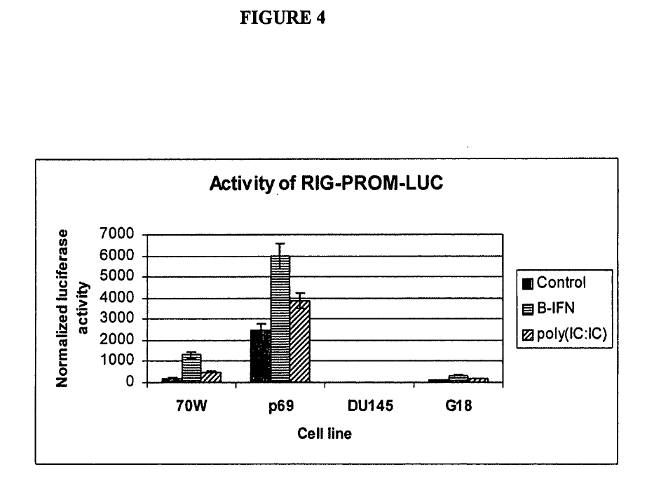

Another panel of cells was tested including 70 W human melanoma, p69 immortalized human prostate epithelial cells, DU-145 prostate carcinoma and G18 glioblastoma derived cells (FIG. 4) utilizing IFN-β and PolyIC:PolyIC as inducer. p69 cells showed maximum induction with IFN-β (3-fold) and lesser induction with PolyIC:PolyIC (2-fold). The overall activity of the promoter was much lower in 70 W melanoma but a statistically significant inducibility over control was observed. G18 and Du-145 cells showed low activity of the reporter (FIG. 4).

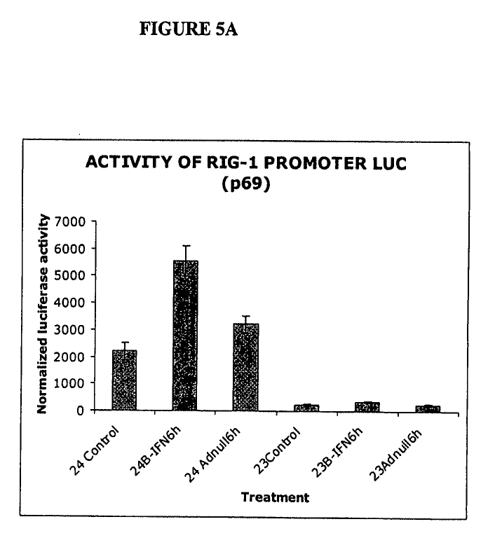

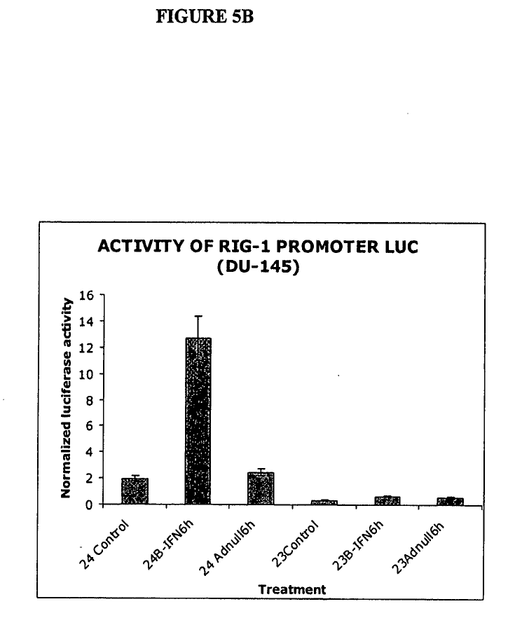

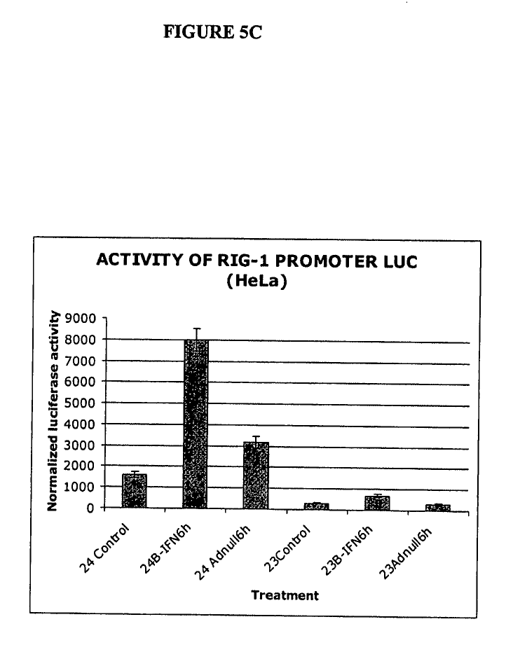

Immortalized normal prostate epithelial cells (p69), human prostate cancer cells (DU-145) and human cervical cancer cells (HeLa) were in three parallel sets of transfections for each cell type with the full length RIG-PROM-LUC reporter and a construct mutated in an Interferon Regulatory Factor-1 (IRF-1) (Malmgaard, Interferon Cytokine Res. 2004 8:439-454) binding site at positions 3950 to 3967 of SEQ ID NO:1. One set of cells was not treated (Control), one set was treated with 2000 units/ml of IFN-β and the third set was infected with 100 pfu of Adnull (a null adenovirus control vector not expressing any exogenous gene) (FIG. 5A, B, C). IFN-β treatment of transfected cells induced full length RIG-PROM-LUC reporter activity between 3-7 fold over control and Adnull infection activated reporter activity between 1.5-2.5 fold (FIG. 5A, B, C). Treatment with either inducer did not affect the activity of mutant IRF-1 RIG-PROM-LUC reporter (FIG. 5A, B, C). The un-induced (Control) activity of mutant IRF-1 RIG-PROM-LUC reporter is extremely low indicating that mutation of the IRF-1 site could negatively affect RIG-PROM-LUC basal as well as activated expression.

From the results described supra there is clear demonstration that a fully active promoter for the human RIG-1 gene has been isolated. The promoter shows activity and inducibility in some cell types and not in others.

Various publications are cited herein, the contents of which are hereby incorporated by reference in their entireties.

1-23. (canceled)

24. An isolated nucleic acid comprising a Retinoic Acid Inducible Gene-1 promoter which is at least 90 percent homologous to SEQ ID NO:1.

25. The isolated nucleic acid of claim 24, which is contained in plasmid RIG-PROM-LUC deposited with the American Type Culture Collection and assigned Accession No. PTA-6251.

26. The isolated nucleic acid of claim 24, wherein the Retinoic Acid Inducible Gene-1 promoter is operably linked to a gene of interest, where the gene of interest is not the Retinoic Acid Inducible Gene-1.

27. The nucleic acid of claim 26, wherein the gene of interest is a reporter gene.

28. The nucleic acid of claim 27, wherein the reporter gene is selected from the group consisting of Luciferase, Green, Cyan, Yellow, Red or Far Red Fluorescent Protein, Secreted Alkaline Phosphatase and β-Galactosidase.

29. A vector containing the nucleic acid of claim 24.

30. A vector containing the nucleic acid of claim 26.

31. A cell containing the nucleic acid of claim 24.

32. A cell containing the nucleic acid of claim 26.

33. A non-human transgenic animal containing a transgene comprising the isolated nucleic acid of claim 3.

34. The non-human transgenic animal of claim 33, wherein the gene of interest is a reporter gene.

35. The non-human transgenic animal of claim 34, wherein the reporter gene is selected from the group consisting of Luciferase, Green, Cyan, Yellow, Red or Far Red Fluorescent Protein, Secreted Alkaline Phosphatase and β-Galactosidase.

36. A method for determining whether a test agent has anti-viral activity, comprising:
(i) providing a cell containing a nucleic acid comprising a Retinoic Acid Inducible Gene-1 promoter operably linked to a reporter gene;
(ii) exposing the cell to the test agent; and
(iii) determining whether exposure to the test agent results in an increase in expression of the reporter gene;
wherein an increase in the expression of the reporter gene indicates that the test agent is an anti-viral agent.

37. A kit containing a nucleic acid comprising a Retinoic Acid Inducible Gene-1 promoter operably linked to a reporter gene and a reporter gene detecting agent.

38. The kit of claim 14, wherein the reporter gene is selected from the group consisting of Luciferase, Green, Cyan, Yellow, Red or Far Red Fluorescent Protein, Secreted Alkaline Phosphatase and β-Galactosidase. and the detecting agent is an antibody.

39. The kit of claim 37, wherein the reporter gene is Luciferase and the detecting agent is luciferin and ATP in a suitable reaction buffer.

40. The kit of claim 37, wherein the reporter gene is a fluorescent protein including Green, Cyan, Yellow, Red or Far Red Fluorescent Protein and the detecting agent is visualization by fluorescence or confocal microscopy.

41. The kit of claim 37, wherein the reporter gene is a fluorescent protein including Green, Cyan, Yellow, Red or Far Red Fluorescent Protein and the detecting agent is visualization by a microtitration plate reader capable of detecting fluorescent signals.

42. An isolated nucleic acid comprising a a Retinoic Acid Inducible Gene-1 promoter, obtained by using a primer selected from the group of nucleic acid molecules defined by SEQ ID NO:2 and SEQ ID NO:3 in a polymerase chain reaction with normal human genomic DNA as template.

43. A method of constructiong a Retinoic Acid Inducible Gene-1 promoter comprising amplifying the promoter from a nucleic acid using one or both of the primers defined by SEQ ID NO:2 and SEQ ID NO:3.
